(19) United States
(12) Patent Application Publication
(10) Pub. No.: US 2023/0263178 A1
(43) Pub. Date: Aug. 24, 2023

(54) BOVINE MILK EXOSOME PRODUCTS AND METHODS, NUTRITIONAL COMPOSITIONS, AND THERAPEUTIC METHODS

(71) Applicant: Abbott Laboratories, Abbott Park, IL (US)

(72) Inventors: José María LÓPEZ PEDROSA, Cenes de la Vega (ES); Ricardo RUEDA CABRERA, Granada (ES); Jorge GARCÍA MARTÍNEZ, Armilla (ES)

(21) Appl. No.: 17/907,750

(22) PCT Filed: Apr. 1, 2021
(86) PCT No.: PCT/US2021/025300

§ 371 Date: Sep. 29, 2022

(30) Foreign Application Priority Data
Apr. 3, 2020 (EP) 20382267
Oct. 27, 2020 (EP) 20382934

(51) Int. Cl. A23C 9/142; A23L 33/00; A61P 3/10; A61K 35/20

(52) U.S. Cl. CPC A23C 9/1425; A23L 33/40; A61P 3/10; A61K 35/20

(57) ABSTRACT

A method of obtaining an exosome-enriched product comprises providing a whey-containing bovine milk fraction, conducting a first centrifugation of the whey-containing bovine milk fraction to obtain a whey middle fraction, conducting a second centrifugation of the whey middle fraction at an increased speed to obtain a concentrated whey fraction, filtering the concentrated whey fraction to obtain a filtered whey fraction, and conducting a third centrifugation of the filtered whey fraction at a further increased speed to obtain an exosome-enriched product. The exosome-enriched product comprises intact exosomes and less than 5 wt % casein based on the total weight of protein in the exosome-enriched product. Nutritional compositions comprise protein, carbohydrate, and/or fat, and exosomes provided by addition of the exosome-enriched product. A method of improving insulin sensitivity in a subject comprises administering a nutritional composition comprising the exosome-enriched product.

### FIELD OF THE INVENTION

The present invention relates to methods of obtaining an exosome-enriched product from a whey-containing bovine milk fraction or, specifically, from cheese whey. The present invention also relates to an exosome-enriched product, to nutritional compositions containing bovine milk-derived exosomes, and methods of improving insulin sensitivity in a subject.

### BACKGROUND OF THE INVENTION

Bovine milk contains exosomes which are extracellular membrane vesicles. By way of example, bovine milk exosomes contain bioactive agents including, but not limited to, enzymatic and non-enzymatic proteins (CD9, CD63, MHC-class II, lactadherin, TSG101 and Hsc70, among others.), nucleic acids (including high amounts of microRNA (miRNA) and messenger RNA (mRNA)) and lipids (phosphatidylethanolamine, phosphatidylserine, phosphatidylcholine and sphingomyelin, among others), that are known to have a role in regulating health and disease. Milk exosomes can be actively taken up by cells to deliver their content, but uptake requires the milk exosome vesicle membrane to be preserved.

While the exosome structure protects bioactive agents, known methods of isolation of exosomes from milk can damage the exosome membrane, preventing uptake of the exosomes and resulting in release of bioactive agents and degradation of their bioactivity. Thus, although miRNAs play an important role in regulating health and disease, food-borne miRNAs have not been explored as potential functional ingredients by the nutrition industry, since isolated miRNAs are extremely unstable and may degrade within seconds.

In addition to the above concerns, there are other technical difficulties that arise when isolating exosomes from milk products. For example, milk exosomes are nanosized particles and are typically 10 to 200 nm in size. Caseins, which account for nearly 80% of milk proteins, have a tendency to form colloidal aggregates that are around 20 to 600 nm in size. The size of exosomes and casein aggregates thus overlaps. In view of this overlap, size-based isolation techniques, which are common in the dairy industry, tend to co-purify exosomes and casein. Accordingly, improved methods of isolating bovine milk exosomes are desirable.

Insulin sensitivity resistance, prediabetes, and diabetes are conditions of glucose dysregulation. Lifestyle modifications and insulin sensitizers are among the treatment strategies available, however these often fail and insulin therapy can become necessary. Dietary management can improve metabolic health and lead to weight loss. Planning for weight maintenance over time, sustaining and improving muscle mass, and taking vitamins and minerals can improve quality of life. Unfortunately, in many cases, lifestyle modifications are inadequate to fully treat such conditions. New compositions and methods for improving insulin sensitivity are therefore needed in order to treat insulin resistance, prediabetes, and diabetes.

### SUMMARY OF THE INVENTION

In one embodiment, the invention is directed to a method of obtaining an exosome-enriched product from a bovine milk fraction. The method comprises providing a whey-containing bovine milk fraction, a first centrifugation which comprises centrifuging the whey-containing bovine milk fraction to obtain a whey middle fraction, a second centrifugation which comprises centrifuging the whey middle fraction at an increased speed as compared with the speed of the first centrifugation to obtain a concentrated whey fraction, filtering the concentrated whey fraction to obtain a filtered whey fraction, and a third centrifugation which comprises centrifuging the filtered whey fraction at an increased speed as compared with the speed of the second centrifugation to obtain an exosome-enriched product, wherein the exosome-enriched product comprises intact exosomes and less than 5 wt % casein based on the total weight of protein in the exosome-enriched product.

In a more specific embodiment, the invention is directed to a method of obtaining an exosome-enriched product from cheese whey. The method comprises a first centrifugation comprising centrifuging cheese whey to obtain a whey middle fraction, a second centrifugation comprising centrifuging the whey middle fraction at an increased speed as compared with the speed of the first centrifugation to obtain a concentrated whey fraction, filtering the concentrated whey fraction to obtain a filtered whey fraction, and a third centrifugation comprising centrifuging the filtered whey fraction at an increased speed as compared with the speed of the second centrifugation to obtain an exosome-enriched product, wherein the exosome-enriched product comprises intact exosomes and less than 5 wt % casein based on the total weight of protein in the exosome-enriched product.

In an additional embodiment, the invention is directed to a bovine milk-derived exosome-enriched product comprising intact exosomes and less than 5 wt % casein based on the total weight of protein in the exosome-enriched product. In a further embodiment, the invention is directed to an exosome-enriched product obtained by a method as described herein.

In another embodiment, the invention is directed to a nutritional composition which comprises protein, carbohydrate, and/or fat, and bovine milk-derived exosomes, wherein the exosomes are provided by addition of the exosome-enriched product of the invention. In specific embodiments, the nutritional composition is in the form of either a powder or a liquid.

In further embodiments, the invention is directed to a method of improving insulin sensitivity in a subject. The method comprises administering to a subject at risk of developing, or having, insulin resistance, prediabetes, or diabetes, the exosome-enriched product of the invention or a nutritional composition comprising the exosome-enriched product of the invention.

The methods of obtaining an exosome-enriched product of the present invention are advantageous in that they are suitable for industrialized enrichment of milk exosomes, and further in that they are able to reduce the costs associated with other milk exosome isolation and enrichment techniques, particularly if a whey by-product from cheese making is employed as a starting material in the inventive methods. Furthermore, in certain embodiments, the methods of the invention provide an improved exosome-enriched product since casein, which has a size similar to that of milk exosomes, is or has already been substantially removed from the cheese whey or whey-containing bovine milk fraction source. In addition, the exosome-enriched products of the invention are advantageous in providing intact exosomes comprising bioactive agents, such as miRNAs, in a convenient and stable form, and, in certain embodiments, in a nutritional composition. The use of the enriched exosome products and nutritional compositions for therapeutic benefits, for example for improving insulin sensitivity, is therefore facilitated. These and additional objects and advantages of the invention will be more fully apparent in view of the following detailed description.

### BRIEF DESCRIPTION OF THE DRAWING

The drawing is illustrative of certain embodiments of the invention and exemplary in nature and are not intended to limit the invention defined by the claims, wherein:

FIG. 1 illustrates glucose uptake in muscle cells incubated with increasing concentrations of freeze-dried cheese whey exosomes for either 5 or 24 hours, as described in Example 2.

### DETAILED DESCRIPTION

Specific embodiments of the invention are described herein. The invention can, however, be embodied in different forms and should not be construed as limited to the embodiments set forth herein. Rather, these embodiments are provided to illustrate more specific features of certain embodiments of the invention to those skilled in the art.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

To the extent that the term “includes” or “including” is used in the description or the claims, it is intended to be inclusive of additional elements or steps, in a manner similar to the term “comprising” as that term is interpreted when employed as a transitional word in a claim. Furthermore, to the extent that the term “or” is employed (e.g., A or B), it is intended to mean “A or B or both.” When the “only A or B but not both” is intended, then the term “only A or B but not both” is employed. Thus, use of the term “or” herein is the inclusive, and not the exclusive use. When the term “and” as well as “or” are used together, as in “A and/or B” this indicates A or B as well as A and B.

The exosome-enriched products, the nutritional compositions, and the methods described in the present disclosure can comprise, consist of, or consist essentially of any of the elements and steps as described herein.

All ranges and parameters, including but not limited to percentages, parts, and ratios disclosed herein are understood to encompass any and all sub-ranges subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, and 10) contained within the range.

Any combination of method or process steps as used herein can be performed in any order, unless otherwise specified or clearly implied to the contrary by the context in which the referenced combination is made.

All percentages are percentages by weight unless otherwise indicated.

The term “bovine milk-derived exosomes” as used herein, unless otherwise specified, refers to exosomes that have been substantially separated from other bovine milk components such as lipids, cells, and debris, and are concentrated in an amount higher than that found in bovine milk. The exosomes are small, extracellular vesicles and account for a minor percentage of milk's total content. Enrichment of the exosomes as described herein produces a product in which the exosomes originally present in milk are concentrated.

The term “exosome-enriched powder” as used herein, unless otherwise specified, refers to a dry powder that contains exosomes which have been isolated from a bovine milk fraction. The exosomes are dried to form a dry powder. In specific examples, the isolated exosomes have been freeze-dried to form the exosome-enriched powder.

The term “exosome-enriched product” as used herein, unless otherwise specified, refers to a product that contains exosomes which have been isolated from a bovine milk fraction. In specific examples, the isolated exosomes have been freeze-dried to form the exosome-enriched product.

Importantly, the exosome-enriched products of the invention comprise intact exosomes. An intact exosome is one in which the vesicle membrane is undamaged and the contents of the exosome, for example functional lipids (e.g., gangliosides, sphingomyelin, phosphatidylcholine, phosphatidylethanolamine), proteins (e.g., lactoferrin, immunoglobulins), and regulatory miRNAs (e.g., miR-23a, miR-26a, miR-26b, miR-27b), are retained within the exosome.

As previously mentioned, milk contains exosomes which contain bioactive agents that can improve health. For example, bovine milk contains multiple miRNAs for promoting healthy function of diverse organs, tissues, and systems ranging from muscle to bone and fat to skin, brain, liver, gut, and the vasculature. More specifically, regulatory miRNAs, including miR23a, miR-26a, miR-26b, and miR-27b, are known to play a role in muscle function and glucose metabolism regulation. However, miRNAs tend to degrade quickly, and such beneficial functions are lost. Milk exosomes provide a protective environment for miRNAs, but current techniques for isolating exosomes often lead to damage to the exosome membrane. Moreover, existing techniques used for isolating exosomes from milk are difficult to implement on an industrial scale.

Additionally, known milk exosome isolation techniques tend to co-purify casein and exosomes. Thus, there exists a need for a method of isolating exosomes from milk that provides exosome-enriched products that contain caseins at very low levels, for example less than 5, 4, 3, 2, 1, 0.5, 0.1 wt %, or even lower, based on the total weight of protein in the exosome-enriched product. As mentioned above, various milk solids, such as casein, are similar in size with milk exosomes and are thus co-isolated with milk exosomes when employing existing enrichment techniques. The present invention provides methods that not only reduce co-isolation of casein with exosomes, but also yield an exosome-enriched product comprising a significant amount of intact exosomes. Even further, the methods of the invention are suitable for use on an industrial scale.

Generally, the exosome-enriched products of the invention are obtained from a whey-containing bovine milk fraction source or, more specifically, from a cheese whey source using gentle procedures which do not disrupt the exosome vesicle membrane, thereby leaving the exosome intact and active bioactive agents contained within the exosome structure.

In one embodiment, a whey-containing bovine milk fraction is provided. In a first centrifugation, the whey-containing bovine milk fraction is centrifuged to obtain a whey middle fraction. In a second centrifugation, the whey middle fraction is centrifuged at an increased speed as compared with the speed of the first centrifugation to obtain a concentrated whey fraction, and the concentrated whey fraction is filtered to obtain a filtered whey fraction. In a third centrifugation, the filtered whey fraction is centrifuged at an increased speed as compared with the speed of the second centrifugation to obtain an exosome-enriched product comprising intact exosomes and less than 5 wt % casein based on the total weight of protein in the exosome-enriched product.

In a specific embodiment, the whey-containing bovine milk fraction is provided by lowering the pH of a bovine milk product, typically skim milk, to precipitate milk solids, and removing the milk solids. Such a fraction is often produced as a by-product in cheese-making and referred to as acid whey or acid cheese whey. In another specific embodiment, the pH of the bovine milk fraction is lowered to about 3.0 to 4.6.

When the pH is lowered to about 3.0 to about 4.6, certain milk solids, such as casein, will coagulate. For example, since the isoelectric point of casein is 4.6, lowering the pH of the whey-containing bovine milk fraction to 4.6 will lead to casein precipitation. The casein may then be removed, thereby reducing the amount of casein that will be co-isolated with the exosomes during exosome enrichment. The resulting concentrated whey fraction therefore contains significantly less casein than it would according to typical methods of exosome enrichment straight from bovine milk. Surprisingly, the low pH condition which is required to remove casein does not disadvantageously affect the integrity of the exosomes in the resulting milk fraction. Thus, the resulting milk fraction contains intact exosomes and typically has a pH of about 4.6.

In another embodiment, cheese whey, sometimes referred to as sweet whey or sweet cheese whey, is used as a starting material. Cheese whey is the liquid by-product of milk after the formation of curd during the cheese-making or casein manufacturing process. Since cheese whey has already been separated from the casein fraction during the cheese manufacture process, cheese whey has very low casein content. Furthermore, cheese whey advantageously retains more than 50% of milk nutrients, including lactose, fat, proteins, mineral salts, and, surprisingly, a significant number of exosomes that were originally present in the milk in intact form. In addition to these benefits, cheese whey is less expensive than raw milk, and thus using cheese whey as a starting material significantly reduces costs for production of an exosome-enriched product. As such, cheese whey is a novel and promising source for isolating milk exosomes and producing exosome-enriched products.

In a specific embodiment, the cheese whey is obtained by applying an enzyme or enzyme mixture, and more specifically a protease enzyme, for example chymosin, to milk to hydrolyze casein peptide bonds, thus allowing for enzymatic coagulation of casein in the milk. Thus, when the protease enzyme cleaves the protein, it causes the casein in the milk to coagulate and form a gel structure. The casein protein gel network and milk fat then contract together and form curd. The resulting liquid that is separated from the curd is often referred to as sweet whey or cheese whey, typically has a pH from about 6.0 to about 6.5, and comprises whey proteins, lactose, minerals, water, fat and other low level components.

As indicated above, it is important that the enzyme or enzyme mixture is capable of destabilizing the casein protein in the milk by cleaving peptides which stabilize the casein protein in the milk. Therefore, any proteolytic enzyme suitable for this purpose may be used to obtain cheese whey. In a preferred embodiment, however, the cheese whey is provided by adding rennet enzyme to bovine milk, resulting in enzymatic coagulation of casein. Rennet enzyme is commonly used in the cheese making process and comprises a set of enzymes which are produced in the stomachs of ruminant mammals. These enzymes normally include chymosin, pepsin, and lipase. The rennet enzyme mix destabilizes the casein protein in the bovine milk by proteolytically cleaving peptides which stabilize the protein in the milk. As indicated above, the casein in the milk coagulates and contracts with milk fat to form the cheese curd. The remaining liquid, i.e., the sweet cheese whey, comprises whey proteins, lactose, minerals, water, fat, and other low level components.

In another specific embodiment, cheese whey is first centrifuged to obtain a whey middle fraction. In a second centrifugation, the whey middle fraction is centrifuged at least two times to obtain a concentrated whey fraction, and the concentrated whey fraction is filtered to obtain a filtered whey fraction. In a third centrifugation, the filtered whey fraction is centrifuged at an increased speed as compared with the speed of the second centrifugation to obtain an exosome-enriched product comprising intact exosomes and less than 5 wt % casein based on the total weight of protein in the exosome-enriched product.

In other specific embodiments, the exosome-enriched product comprises at least about 10 wt % exosomes. In other specific embodiments, the exosome-enriched product comprises at least about 15, 20, 25, 30, or 35 wt %. In further embodiments, the exosome-enriched product comprises about 10-15, 10-20, 10-25, 10-30, 10-35, 15-20, 15-25, 15-30, 15-35, 20-25, 20-30, 20-35, 25-30, 25-35, or 30-35 wt %. In another embodiment, at least about 50 wt % of the exosomes in the exosome-enriched product are intact. In other specific embodiments, at least about at least about 55, 60, 65, 70, 75, 80, 85, 90, or 95 wt % of the exosomes in the exosome-enriched product are intact.

In further embodiments, the exosome-enriched product comprises less than 4, 3, 2, 1, 0.5, 0.1, or even lower wt % casein, based on the total weight of protein in the exosome-enriched product.

Methods of obtaining an exosome-enriched product of the invention may comprise centrifugation at specific speeds, times and/or temperatures. The first centrifugation of the whey-containing bovine milk fraction or the cheese whey-derived whey-containing fraction, hereinafter referred to as the “whey-containing fraction”, will form a lipid fraction top layer, a whey middle fraction, and a first pellet of cells and debris. In specific embodiments, the whey-containing fraction may be centrifuged, for example, at 12,000 G for about 15 minutes at about 4° C. to remove residual fat, i.e., the lipid fraction top layer, and other debris, i.e., the first pellet of cells and debris. A second centrifugation may also be conducted, which comprises centrifuging the whey middle fraction at an increased speed as compared with the speed of the first centrifugation, to form an upper layer comprising residual fat, a concentrated whey fraction, and a second pellet of other debris. In a specific embodiment, the second centrifugation comprises centrifuging the whey middle fraction at least two times to obtain the concentrated whey fraction.

In further specific embodiments, the whey middle fraction may be centrifuged at 21,500 G for 30 minutes at 4° C. at least two times in order to remove residual fat and other debris. In a specific embodiment, the centrifugations step(s) are conducted until the resulting concentrated whey fraction is substantially clear. In a further embodiment, the concentrated whey fraction is filtered to obtain a filtered whey fraction. In certain embodiments, the step of filtering the concentrated whey fraction may include microfiltration. In this regard, the concentrated whey fraction may be filtered through a microfilter to remove residual debris. Examples of suitable filters include a 0.22 μm polyethersulfone (PES) filter or other comparable hydrophilic, low protein retention filter. Finally, a third centrifugation may be conducted, for example, by centrifuging the filtered whey fraction at an increased speed as compared with the speed of the second centrifugation to obtain an exosome-enriched product, wherein the exosome-enriched product comprises intact exosomes and less than 5 wt % casein based on the total weight of protein in the exosome-enriched product.

In specific embodiments, the third centrifugation comprises ultracentrifuging the filtered whey fraction to obtain a third pellet containing intact exosomes. In certain embodiments, the third centrifugation comprises ultracentrifuging the filtered whey fraction at an increased speed as compared with the speed of the second centrifugation, for example at 100,000 G for 1 hour at 4° C. to pellet the exosomes contained in the filtered whey fraction.

In specific embodiments, the method further comprises incubating the third pellet containing intact exosomes in an aqueous medium in order to dissolve the third pellet without disrupting exosome membranes, thus resulting in an exosome suspension. In certain embodiments, the third pellet may be suspended with the aqueous buffer in a centrifugation tube and incubated for 12-36 hours in, for example, an orbital shaker at 4° C. and 150 rpm. Examples of suitable aqueous suspensions include sterile PBS buffer (137 mM NaCl, 2.7 mM KCl, 8 mM Na₂HPO₄, and 2 mM KH₂PO₄; pH 7.4) or sterile molecular biology grade water. The exosome-enriched product may then be used or, if desired, for later use, frozen at, for example, −80° C. for at least 2 hours.

In specific embodiments, the method of obtaining the exosome-enriched product further comprises drying the exosome suspension. In a further embodiment, the drying step may comprise freeze drying. The step of freeze-drying may comprise exposing the exosome suspension to a temperature of −80° C. and a vacuum of less than 0.3 mbar for a sufficient time period. In specific embodiments, depending on the amount of liquid to be freeze-dried and the features of the freeze-drying equipment, the time may vary, but can be from about 5 to about 40 hours, more specifically from about 10 to about 30 hours, or from about 15 to about 25 hours. Importantly, the process should fully dry the exosomes. In a specific embodiment as described in the examples, at a temperature of −80° C. and a vacuum of less than 0.3 mbar, the time period for freeze-drying is suitably 24 hours or more. Once frozen, it is advantageous to keep the exosomes frozen prior to freeze drying, i.e., avoid thaw-freeze cycling.

In specific embodiments, the exosome-enriched product comprises functional lipids gangliosides, sphingomyelin, phosphatidylcholine, and phosphatidylethanolamine), proteins (i.e., lactoferrin and immunoglobulins), regulatory miRNAs miR-23a, miR-26a, miR-26b, and miR-27b). The exosome-enriched product can comprise additional bioactive molecules, as will be appreciated by a person of skill in the art.

According to another embodiment of the invention, there is provided a bovine milk-derived exosome-enriched product comprising bovine milk-derived intact exosomes and less than 5 wt % casein based on the total weight of protein in the exosome-enriched product. In a specific embodiment, the exosome-enriched product comprises less than 4, 3, 2, 1, 0.5, or 0.1 wt % casein based on the total weight of protein in the exosome-enriched product.

In additional specific embodiments, the bovine milk-derived exosome-enriched product comprises at least about 10 wt % exosomes. In other specific embodiments, the exosome-enriched product comprises at least about 15, 20, 25, 30, or 35 wt %. In further embodiments, the exosome-enriched product comprises about 10-15, 10-20, 10-25, 10-30, 10-35, 15-20, 15-25, 15-30, 15-35, 20-25, 20-30, 20-35, 25-30, 25-35, or 30-35 wt %. According to certain embodiments, at least 50 wt % of the exosomes in the bovine milk-derived exosome-enriched product are intact. In other embodiments, at least about 55, 60, 65, 70, 75, 80, 85, 90, or 95 wt % of the exosomes in the bovine milk-derived exosome-enriched product are intact.

The bovine milk-derived exosome-enriched products of the invention comprise bioactive molecules, such as functional lipids, proteins, and regulatory miRNAs. In specific embodiments, the functional lipids include gangliosides, sphingomyelin, phosphatidylcholine, and phosphatidylethanolamine. In other specific embodiments, the proteins include lactoferrin and immunoglobulins. Regulatory miRNAs include miR-23a, miR-26a, miR-26b, and miR-27b. Again, the bovine milk-derived exosome-enriched product can comprise additional bioactive molecules, as will be appreciated by a person of skill in the art.

In a specific embodiment, the bovine milk-derived exosome-enriched product is a powder. The bovine milk-derived exosome-enriched powder of the invention comprises intact exosomes, exosomes in which the vesicle membrane is not ruptured and/or otherwise degraded and the bioactive contents of the exosomes are retained therein in active form. Such exosome-containing powders exhibit good stability at higher temperature conditions.

In an additional embodiment, a nutritional composition of the invention comprises protein, carbohydrate, and/or fat, and bovine milk-derived exosomes, wherein the exosomes are provided by addition of an exosome-enriched product described herein or by the addition of an exosome-enriched product obtained by the methods described herein. The exosomes may be included in the nutritional compositions in any desired amount effective to provide nutritional or therapeutic benefit. In specific embodiments, the nutritional composition comprises from about 0.001 to about 10 wt % of the exosomes, based on the weight of the nutritional composition.

A wide variety of one or more proteins, carbohydrates, and/or fats can be used in the nutritional composition of the invention. For example, the protein can include intact, hydrolyzed, and/or partially hydrolyzed protein, which can be derived from a suitable source such as milk (e.g., casein, whey), animal (e.g., meat, fish), cereal (e.g., rice, corn), vegetable (e.g., soy, pea), and combinations thereof. More particular examples of protein used in specific embodiments of the nutritional composition include, but are not limited to, whey protein concentrate, whey protein isolate, whey protein hydrolysate, acid casein, sodium caseinate, calcium caseinate, potassium caseinate, casein hydrolysate, milk protein concentrate, milk protein isolate, milk protein hydrolysate, nonfat dry milk, condensed skim milk, soy protein concentrate, soy protein isolate, soy protein hydrolysate, pea protein concentrate, pea protein isolate, pea protein hydrolysate, collagen protein, collagen protein isolate, rice protein, potato protein, earthworm protein, and/or insect protein, and combinations of two or more thereof. Specific embodiments of the invention comprise fava bean protein, whey protein concentrate, whey protein isolate, whey protein hydrolysate, milk protein concentrate, milk protein isolate, milk protein hydrolysate, soy protein concentrate, soy protein isolate, soy protein hydrolysate, pea protein concentrate, pea protein isolate, pea protein hydrolysate, and combinations of two or more thereof.

Examples of suitable carbohydrates include simple, complex, or a combination thereof. Non-limiting examples of carbohydrates suitable for use in a nutritional composition described herein include human milk oligosaccharides (HMOs), maltodextrin, corn maltodextrin, hydrolyzed starch, glucose polymers, corn syrup, corn syrup solids, rice-derived carbohydrates, sucrose, glucose, lactose, honey, sugar alcohols, isomaltulose, sucromalt, sucralose, pullulan, potato starch, galactooligosaccharides, oat fiber, soy fiber, corn fiber, gum arabic, sodium carboxymethylcellulose, methylcellulose, cellulose gel, cellulose gum, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, carrageenan, psyllium, digestion-resistant carbohydrates such as digestion-resistant maltodextrins, digestion-resistant starch, slowly digestible carbohydrates, inulin, fructooligosaccharides, and combinations of two or more thereof. Specific embodiments of the invention comprise human milk oligosaccharides (HMOs), maltodextrin, corn maltodextrin, corn syrup, sucralose, cellulose gel, cellulose gum, gellan gum, carrageenan, fructooligosaccharides, and combinations of two or more thereof.

Examples of suitable fats include one or more of coconut oil, fractionated coconut oil, soy oil (e.g., high oleic soy oil), soy lecithin, corn oil, olive oil, safflower oil (e.g., high oleic safflower oil), medium chain triglyceride oil (MCT oil), high gamma linolenic (GLA) safflower oil, sunflower oil (e.g., high oleic sunflower oil), palm oil, palm kernel oil, palm olein, canola oil (e.g., high oleic canola oil), marine oils, fish oils (e.g., tuna oil), algal oils, borage oil, cottonseed oil, fungal oils, eicosapentaenoic acid (EPA), docosahexaenoic acid (DHA), arachidonic acid (ARA), conjugated linoleic acid (CLA), alpha-linolenic acid, interesterified oils, transesterified oils, structured lipids, and combinations thereof. The source of fat can comprise triglycerides, diglycerides, monoglycerides, phospholipids (e.g., lecithin) and/or free fatty acids. Fatty acids can include, but are not limited to, capric acid, lauric acid, myristic acid, palmitic acid, palmitoleic acid, stearic acid, oleic acid, linoleic acid, alpha-linolenic acid, arachidonic acid (ARA), eicopanteanoic acid (EPA), and/or docosahexanoic acid (DHA). The nutritional composition of the present disclosure can include any individual source of fat or a combination of two or more the various sources of fat listed above. More specific examples include canola oil, high oleic sunflower oil, medium chain triglycerides and/or one or more fatty acids such as linoleic acid, alpha-linolenic acid, ARA, EPA, and/or DHA. Specific embodiments of the invention comprise coconut oil, fractionated coconut oil, soy oil, soy lecithin, corn oil, safflower oil sunflower oil, palm olein, canola oil monoglycerides, lecithin, and combinations of two or more thereof.

In a specific embodiment, the protein source comprises milk protein and/or soy protein. In a further embodiment, the carbohydrate comprises fiber. In another embodiment, the fat comprises at least one omega-3 fatty acid. In a specific embodiment, the at least one omega-3 fatty acid of the composition is selected from the group consisting of eicosapentaenoic acid, docosahexaenoic acid, arachidonic acid, and alpha-linolenic acid.

In specific embodiments of the nutritional composition, protein comprises from about 1 wt % to about 30 wt % of the nutritional composition. In more specific embodiments, the protein comprises from about 1 wt % to about 25 wt % of the nutritional composition, including about 1 wt % to about 20 wt %, about 1 wt % to about 15 wt %, about 1 wt % to about 10 wt %, about 5 wt % to about 10 wt %, or about 10 wt % to about 20 wt % of the nutritional composition. In additional specific embodiments, the protein comprises from about 1 wt % to about 5 wt % of the nutritional composition. In additional, specific embodiments, the protein comprises from about 20 wt % to about 30 wt % of the nutritional composition.

In specific embodiments of the nutritional composition, carbohydrate is present in an amount from about 5 wt % to about 75 wt % of the nutritional composition. In more specific embodiments, the carbohydrate is present in an amount from about 5 wt % to about 70 wt % of the nutritional composition, including about 5 wt % to about 65 wt %, about 5 wt % to about 50 wt %, about 5 wt % to about 40 wt %, about 5 wt % to about 30 wt %, about 5 wt % to about 25 wt %, about 10 wt % to about 65 wt %, about 20 wt % to about 65 wt %, about 30 wt % to about 65 wt %, about 40 wt % to about 65 wt %, or about 15 wt % to about 25 wt %, of the nutritional composition.

In specific embodiments, the nutritional composition comprises fat in an amount of from about 0.5 wt % to about 30 wt % of the nutritional composition. In certain specific embodiments, the fat comprises from about 1 wt % to about 30 wt % of the nutritional composition, including about 1 wt % to about 20 wt %, about 1 wt % to about 15 wt %, about 1 wt % to about 10 wt %, about 1 wt % to about 5 wt %, about 3 wt % to about 30 wt %, about 5 wt % to about 30 wt %, about 5 wt % to about 25 wt %, about 5 wt % to about 20 wt %, about 5 wt % to about 10 wt %, or about 10 wt % to about 20 wt % of the nutritional composition.

In a specific embodiment, the nutritional composition may be in the form of a powder or a liquid.

In specific embodiments, when the nutritional composition is a liquid, for example, reconstituted from a powder as described herein or manufactured as a ready-to-drink product, a serving ranges from about 1 ml to about 500 ml, including from about 110 ml to about 500 ml, from about 110 ml to about 417 ml, from about 120 ml to about 500 ml, from about 120 ml to about 417 ml, from about 177 ml to about 417 ml, from about 207 ml to about 296 ml, from about 230 m to about 245 ml, from about 110 ml to about 237 ml, from about 120 ml to about 245 ml, from about 110 ml to about 150 ml, and from about 120 ml to about 150 ml. In specific embodiments, the serving is about 1 ml, or about 100 ml, or about 225 ml, or about 237 ml, or about 500 ml.

In specific embodiments, when the nutritional composition is a powder, for example, a serving size is from about 40 g to about 60 g, such as 45 g, or 48.6 g, or 50 g, to be administered as a powder or to be reconstituted in from about 1 ml to about 500 ml of liquid, such as about 225 ml, or from about 230 ml to about 245 ml.

Additional specific embodiments of the nutritional composition comprise one or more components to modify the physical, chemical, aesthetic, or processing characteristics of the nutritional composition or serve as additional nutritional components. Non-limiting examples of additional components include preservatives, emulsifying agents (e.g., lecithin), buffers, sweeteners including artificial sweeteners (e.g., saccharine, aspartame, acesulfame K, sucralose), colorants, flavorants, thickening agents, stabilizers, and so forth.

Specific embodiments of the nutritional composition may comprise vitamins and/or related nutrients, non-limiting examples of which include vitamin A, vitamin B12, vitamin C, vitamin D, vitamin E, vitamin K, thiamine, riboflavin, pyridoxine, niacin, folic acid, pantothenic acid, biotin, choline, inositol, and/or salts and derivatives thereof, and combinations thereof.

Specific embodiments of the nutritional composition comprise minerals, non-limiting examples of which include calcium, phosphorus, magnesium, zinc, manganese, sodium, potassium, molybdenum, chromium, iron, copper, and/or chloride, and combinations thereof.

Due to the intact nature of the exosomes in the powdered form, the exosomes, and compositions to which the powdered exosomes are added, are suitable for use in providing a nutritional or therapeutic benefit based on the bioactive agents in the exosomes, including various miRNA contained in intact exosomes. In specific embodiments, the powdered exosomes, and compositions to which the powdered exosomes are added, are suitable for use in lowering a risk of insulin resistance, prediabetes, or diabetes, and/or for treating insulin resistance, prediabetes, or diabetes. Risk factors for developing insulin resistance, prediabetes, or diabetes can include being overweight, being sedentary or physically active less than 3 times per week, having had gestational diabetes previously, or having an immune disorder. Subjects with insulin resistance, prediabetes, or diabetes are also often obese, hypertensive, have dyslipidemia, and/or have sarcopenia.

In specific embodiments, the methods for lowering a risk of insulin resistance, prediabetes, or diabetes, or treating these conditions, comprise administering to a subject the bovine milk-isolated powdered exosomes as described herein or a nutritional composition to which the bovine milk-isolated powdered exosomes as described herein have been added. The nutritional composition may be administered in powder or liquid form, as desired. In specific embodiments, the bovine milk-isolated powdered exosomes or nutritional compositions to which the bovine milk-isolated powdered exosomes as described herein have been added are administered to a subject once or multiple times daily or weekly. In specific embodiments, the nutritional composition is administered to the subject from about 1 to about 6 times per day or per week, or from about 1 to about 5 times per day or per week, or from about 1 to about 4 times per day or per week, or from about 1 to about 3 times per day or per week.

In specific embodiments, the powdered exosomes of the invention are administered, directly or via addition to a nutritional composition, in an effective amount to lower the risk of insulin resistance, prediabetes and/or diabetes in a subject who is at risk of developing one or more of such conditions. In additional specific embodiments, the powdered exosomes of the invention are administered, directly or via a nutritional composition, in an effective amount to treat insulin resistance, prediabetes and/or diabetes in a subject who is diagnosed with one or more of such conditions. In specific embodiments of the methods described herein, a dosage of from about 0.01 to about 10 g of powdered exosomes is administered to a subject, directly or via addition to a nutritional composition. In additional embodiments, a dosage of from about 0.1 to about 10 g, or from about 1 to about 5 g, of powdered exosomes are administered to a subject, directly or via addition to a nutritional composition.

The following Examples demonstrate various embodiments of the invention.

### EXAMPLES

### Example 1: Cheese Whey Exosome Product and Characterization

This example describes a method of preparing an exosome-enriched product from cheese whey, and characterization of the resulting product. The cheese whey was provided by adding rennet enzyme to bovine milk, resulting in enzymatic coagulation of casein and production of sweet cheese whey, as described above.

Upon reception at 4° C., the cheese whey was aliquoted and immediately frozen at 80° C. Aliquots were thawed on ice and centrifuged at 12,000 G for 15 minutes at 4° C. The upper fraction (i.e., lipid fraction top layer) and the pellet (i.e., first pellet of cells and debris) were discarded and the middle fraction (i.e., whey middle fraction) was transferred to a clean tube. The whey middle fraction was centrifuged two times at 21,500 G for 30 minutes at 4° C. to remove residual fat (upper layer) and other debris (second pellet). The concentrated clear whey fraction that was obtained from the second centrifugation of the whey middle fraction was filtered through a 0.22 micrometers (μm) polyethersulfone (PES) filter (hydrophilic, low protein retention) to remove residual debris. The resulting filtrate was ultracentrifuged at 100,000 G for 1 hour at 4° C. to produce a third pellet containing exosomes.

Following isolation, the exosomes were resuspended in either sterile PBS buffer (137 mM NaCl, 2.7 mM KCl, 8 mM Na₂HPO₄, and 2 mM KH₂PO₄; pH 7.4) or sterile molecular biology grade water in a centrifugation tube. In order to dissolve the pellet without disrupting the cheese whey exosome membrane, the sterile PBS buffer or sterile molecular biology grade water was added to the centrifugation tube and the pellet in buffer/water was incubated for 12-36 hours in an orbital shaker at 4° C. and 150 rpm. Once dissolved, the exosome-enriched product was frozen at −80° C. for at least 2 hours. The frozen products were subsequently freeze-dried in a Telstar Cryodos-80 freeze dryer at −80° C. and <0.3 mbar for at least 24 hours to 48 hours. The frozen products were not thawed prior to freeze-drying.

Following the step of freeze-drying, the exosome product was resuspended in liquid and analyzed to determine the protein, lipid, and miRNA composition of the exosomes.

As indicated in the preceding paragraph, the exosomes were analyzed in terms of lipid composition. Ultra-performance liquid chromatography coupled to time-of-flight mass spectrometry analysis (UPLC-TOF-MS) was performed to analyze the lipid content of the exosome product isolated by ultracentrifugation-filtration at lab scale. Samples were run by triplicate. The results of predominant bioactive lipids are set forth in Table 1 and demonstrate that the exosome-enriched product contains significant amounts of functional lipids, including gangliosides, sphingomyelin, and phosphatidylcholine. The results are expressed as the % of total lipids.

TABLE 1
Lipid composition of cheese whey exosomes.

| | ABUNDANCE |
|---|---|
| LIPID | (% OF TOTAL LIPIDS) |
| Phosphatidylcholine | 9.0 |
| Sphingomyelin | 3.1 |
| Phosphatidylethanolamine | 2.4 |
| Phosphatidylserine | 1.2 |
| Gangliosides (GD3) | 0.4 |
| Phosphatidylinositol | 0.1 |
| Lysophosphatidylethanolamine | 0.1 |
| Lysophosphatidylcholine | 0.1 |

The protein composition of the exosome product was also analyzed. The protein composition was determined by LC-MS/MS and mass spectra were searched in Proteome Discoverer v1.4 (database Bos Taurus, Uniprot 06/19+ Proteomics contaminants database). The results of several proteins of interest are set forth in Table 2 and surprisingly demonstrate that caseins were present at very low levels (e.g., only 0.04% of a α-S2-casein was detected). In addition, the results demonstrate that significant amounts of bioactive proteins (i.e., lactoferrin and immunoglobulins) were detected. The results are expressed as % of total proteins identified. Samples were run by triplicate.

TABLE 2
Protein composition of cheese whey exosomes.

| | ABUNDANCE |
|---|---|
| PROTEIN | (% OF TOTAL PROTEINS) |
| Xanthine oxidase | 4.5 |
| Lactoferrin | 3.2 |
| Immunoglobulin light chain, | 1.1 |
| lambda gene cluster | |
| a-S2-casein | 0.04 |

The miRNA composition of the exosome product was also analyzed. The exosome-borne miRNAs were sequenced. Total RNA was isolated and purity and integrity were briefly checked. After testing for RNA quality, multiplexed microRNA libraries for Illumina-based sequencing were prepared and sequenced on an Illumina NextSeq 500 apparatus. The resulting sequences were processed and searched in miRBase (Bos Taurus 5.0.1 database). The results of several miRNA of interest, with sequences identical to those of their human counterparts, are set forth in Table 3 and are expressed as % of total miRNAs identified in the sample. The exosome-enriched products contain relatively high levels of these miRNAs. Samples were run by triplicate.

TABLE 3
miRNA profile of the cheese whey exosome fraction.

| | ABUNDANCE |
|---|---|
| miRNA | (% OF TOTAL miRNA) |
| miR-26a | 7.88417 |
| miR-26b | 1.11332 |
| miR23a | 0.47751 |
| miR-27b | 0.12376 |

### Example 2: Therapeutic Results

This example demonstrates that exosome-enriched products comprising intact exosomes and less than 5 wt % casein based on the total weight of protein in the exosome-enriched product, prepared as described above, are able to increase glucose uptake in muscle cells in a dose dependent manner. This was shown by demonstrating an in vitro dose-dependent enhancement of glucose uptake in L6.C11 skeletal muscle cells.

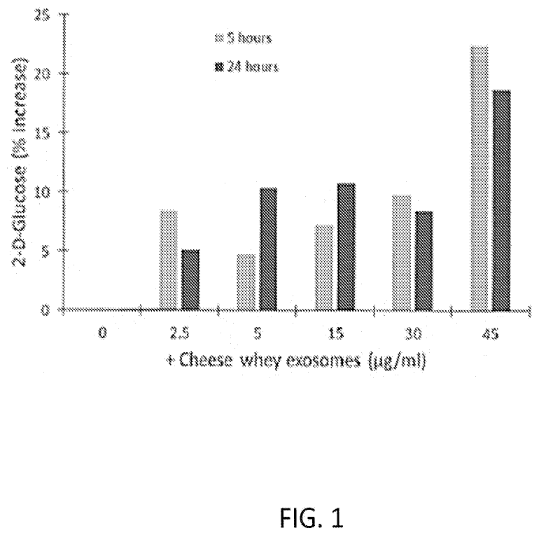

L6.C11 differentiated muscle cells were grown in 48-well plates (Corning, N.Y., USA). Cells were incubated with different concentrations of freeze-dried exosomes (0-45 μg/mL) and incubated for either 5 or 24 hours. Cells were then rinsed with KRPH (HEPES buffered Krebs-Ringer phosphate), consisting of 118 mmol/L NaCl, 5 mmol/L KCl, 1.3 mmol/L CaCl₂), 1.2 mmol/L MgSO₄, 1.2 mmol/L KH₂PO₄, and 30 mmol/L HEPES (pH 7.4). Cells were then immediately incubated with 2-deoxy-[3H]D-glucose (2-DG, radiolabeled glucose) for 10 minutes by triplicate. The uptake of 2-DG was terminated after 10 minutes by rapidly aspirating off the radioactive incubation medium and washing the cells three times with ice-cold phosphate-buffered saline. The radioactivity associated with the cells was determined by cell lysis in 0.5 N NaOH and neutralization with 0.5 N HCl, followed by liquid scintillation. Aliquots from each well were used to determine the protein concentration by BCA Protein assay. The radioactivity associated to the cells was measured and the results are set forth in FIG. 1. The results demonstrate that, when compared to control (i.e., 0 μg/mL exosomes added), muscle cells that were incubated with exosomes for both 5 and 24 hours efficiently increased their glucose uptake rates.

As evidenced by the results in FIG. 1, the exosome-enriched products according to the invention are able to increase glucose uptake in a dose dependent manner. By increasing glucose uptake in muscle cells, it is possible to enhance the control of glucose levels and muscle function in insulin resistant and diabetic subjects by improving insulin sensitivity.

In summary, the exosome-enriched products of the invention provide several advantages. The exosome-enriched products contain high levels of certain miRNAs whose sequence is identical to that of their human counterparts, including, for example, those listed in Table 3. In addition to containing miRNAs, the exosome-enriched products of the invention carry other functional molecules, including bioactive lipids and proteins. Further, the exosome-enriched products of the invention are able to be taken up by muscle cells and are able to increase glucose uptake in a dose dependent manner, and in doing so, provide a method for enhancing control of glucose levels and muscle function in insulin resistant, prediabetic, and diabetic patients by improving insulin sensitivity. Therefore, in specific embodiments, the exosome-enriched products and/or a nutritional composition containing the exosome-enriched products may be used to improve insulin sensitivity in a subject.

The specific embodiments and examples described herein are exemplary only and are not limiting to the invention defined by the claims.

1. A method of obtaining an exosome-enriched product, the method comprising:
providing a whey-containing bovine milk fraction;
conducting a first centrifugation, comprising centrifuging the whey-containing bovine milk fraction to obtain a whey middle fraction;
conducting a second centrifugation, comprising centrifuging the whey middle fraction at an increased speed as compared with the speed of the first centrifugation to obtain a concentrated whey fraction;
filtering the concentrated whey fraction to obtain a filtered whey fraction; and
conducting a third centrifugation, comprising centrifuging the filtered whey fraction at an increased speed as compared with the speed of the second centrifugation to obtain an exosome-enriched product, wherein the exosome-enriched product comprises intact exosomes and less than 5 wt % casein based on the total weight of protein in the exosome-enriched product.

2. The method of claim 1, wherein the whey-containing bovine milk fraction is provided by lowering the pH of a bovine milk product to precipitate milk solids, and removing the milk solids.

3. The method of claim 2, wherein the pH of the milk product is lowered to about 3.0 to 4.6.

4. The method of claim 1, wherein the whey-containing bovine milk fraction comprises.

5. The method of claim 4, wherein the cheese whey is sweet cheese whey and has a pH from about 6.0 to about 6.5.

6. The method of claim 5, wherein the sweet cheese whey comprises liquid separated from curd formed by subjecting bovine milk to at least one enzyme which destabilizes casein.

7. The method of claim 1, wherein the second centrifugation comprises centrifuging the whey middle fraction at least two times to obtain the concentrated whey fraction.

8. The method of claim 1, wherein the exosome-enriched product comprises at least 10 wt % exosomes.

9. The method of claim 8, wherein at least about 50 wt % of exosomes in the exosome-enriched product are intact.

10. The method of claim 9, wherein at least about 55, 60, 65, 70, 75, 80, 85, 90, or 95 wt % of the exosomes in the exosome-enriched product are intact.

11. The method of claim 1, wherein the exosome-enriched product comprises less than 4, 3, 2, 1, 0.5, or 0.1 wt % casein based on the total weight of protein in the exosome-enriched product.

12. The method of claim 1, wherein the third centrifugation comprises ultracentrifuging the filtered whey fraction to obtain a pellet containing intact exosomes.

13. The method of claim 12, further comprising incubating the pellet containing exosomes in aqueous medium to dissolve the pellet without disrupting exosome membranes to provide an exosome suspension.

14. The method of claim 13, further comprising drying the exosome suspension.

15. The method of claim 14, wherein the drying comprises freeze drying the exosome suspension.

16. The method of claim 1, wherein the exosome-enriched product is an exosome-enriched powder.

17. An exosome-enriched product obtained by the method of claim 1.

18. A bovine milk-derived exosome-enriched product comprising bovine milk-derived intact exosomes and less than 5 wt % casein based on the total weight of protein in the exosome-enriched product.

19. The bovine milk-derived exosome-enriched product of claim 18, wherein the exosome-enriched product comprises less than 4, 3, 2, 1, 0.5, or 0.1 wt % casein based on the total weight of protein in the exosome-enriched product.

20. The bovine milk-derived exosome-enriched product of claim 19, wherein the exosome-enriched product comprises at least 10 wt % exosomes.

21. The bovine milk-derived exosome-enriched product of claim 20, wherein at least about 50 wt % of exosomes in the exosome-enriched product are intact.

22. The bovine milk-derived exosome-enriched product of claim 21, wherein at least about 55, 60, 65, 70, 75, 80, 85, 90, or 95 wt % of the exosomes in the exosome-enriched product are intact.

23. The bovine milk-derived exosome-enriched product of claim 22, wherein the exosome-enriched product is a powder.

24. The bovine milk-derived exosome-enriched product of claim 18, wherein the exosomes are included in a nutritional composition comprising protein, carbohydrate, and/or fat, and the exosomes.

25. The nutritional composition of claim 24, comprising from about 0.001 to about 10 wt % of the bovine milk-derived exosomes, based on the weight of the nutritional composition.

26. The nutritional composition of claim 24, wherein the protein comprises whey protein concentrate, whey protein isolate, whey protein hydrolysate, milk protein concentrate, milk protein isolate, milk protein hydrolysate, soy protein concentrate, soy protein isolate, soy protein hydrolysate, pea protein concentrate, pea protein isolate, pea protein hydrolysate, fava bean protein, or combinations of two or more thereof.

27. The nutritional composition of claim 24, wherein the carbohydrate comprises fiber, human milk oligosaccharides (HMOs), maltodextrin, corn maltodextrin, corn syrup, sucralose, cellulose gel, cellulose gum, gellan gum, carrageenan, fructooligosaccharides, or combinations of two or more thereof.

28. The nutritional composition of any one of claim 24, wherein the fat comprises coconut oil, fractionated coconut oil, soy oil, soy lecithin, corn oil, safflower oil sunflower oil, palm olein, canola oil monoglycerides, lecithin, at least one omega-3 fatty acid, anola oil, medium chain triglycerides, one or more fatty acids such as linoleic acid, alpha-linolenic acid, ARA, EPA, and/or DHA, or combinations of two or more thereof.

29. The nutritional composition of claim 28, wherein the at least one omega-3 fatty acid of the composition is selected from the group consisting of eicosapentaenoic acid, docosahexaenoic acid, arachidonic acid, and alpha-linolenic acid.

30. The nutritional composition of claim 29, wherein the nutritional composition is in the form of a powder.

31. The nutritional composition of claim 29, wherein the nutritional composition is in the form of a liquid.

32. A method of improving insulin sensitivity in an individual at risk of developing or having insulin-resistance, prediabetes, or diabetes, comprising:
administering to the individual a nutritional composition comprising the exosome-enriched product of claim 18.
